SEQUENCE LISTING (23 sequences)

SEQ ID NO: 1 - protein, 866 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGDSEHKLETSSGRVLGRFRGKGSGGLNLGNFFASRKGYSRKGFDRLSTEGSDQEKEDDGSESEEEYSAPLPALAPSSS

SEQ ID NO: 2 - protein, 780 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKGKGSGGLNLGNFFASRKGYSRKGFDRLSTEGSDQEKEDDGSESEEEYSAPLPALAPSSS

SEQ ID NO: 3 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 15 aa, Artificial Sequence
  G4Sx3
GGGGSGGGGSGGGGS

SEQ ID NO: 5 - protein, 34 aa, Homo sapiens
IKPEAPGEDASPEELNRYYASLRHYLNLVTRQRY

SEQ ID NO: 6 - protein, 35 aa, Artificial Sequence
  PYY3-36-G
IKPEAPGEDASPEELNRYYASLRHYLNLVTRQRYG

SEQ ID NO: 7 - protein, 36 aa, Artificial Sequence
  PYY3-36-GK
IKPEAPGEDASPEELNRYYASLRHYLNLVTRQRYGK

SEQ ID NO: 8 - protein, 37 aa, Artificial Sequence
  PYY3-36-GKR
IKPEAPGEDASPEELNRYYASLRHYLNLVTRQRYGKR

SEQ ID NO: 9 - protein, 275 aa, Artificial Sequence
  Human IgG1-Fc-G4Sx3-PYY3-36
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIKPEAPGEDASPEELNRYYASLRHYLNLVTRQRY

SEQ ID NO: 10 - protein, 276 aa, Artificial Sequence
  Human IgG1-Fc-G4Sx3-PYY3-36-G
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIKPEAPGEDASPEELNRYYASLRHYLNLVTRQRYG

SEQ ID NO: 11 - protein, 277 aa, Artificial Sequence
  Human IgG1-Fc-G4Sx3-PYY3-36-GK
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIKPEAPGEDASPEELNRYYASLRHYLNLVTRQRYGK

SEQ ID NO: 12 - protein, 278 aa, Artificial Sequence
  Human IgG1-Fc-G4Sx3-PYY3-36-GKR
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIKPEAPGEDASPEELNRYYASLRHYLNLVTRQRYGKR

SEQ ID NO: 13 - protein, 454 aa, Homo sapiens
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 502 aa, Artificial Sequence
  huIgG1-G4Sx3-PYY3-36
VQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIKPEAPGEDASPEELNRYYASLRHYLNLVTRQRY

SEQ ID NO: 15 - protein, 504 aa, Artificial Sequence
  huIgG1-G4Sx3-PYY3-36-G
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIKPEAPGEDASPEELNRYYASLRHYLNLVTRQRYG

SEQ ID NO: 16 - protein, 505 aa, Artificial Sequence
  huIgG1-G4Sx3-PYY3-36-GK
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIKPEAPGEDASPEELNRYYASLRHYLNLVTRQRYGK

SEQ ID NO: 17 - protein, 506 aa, Artificial Sequence
  huIgG1-G4Sx3-PYY3-36-GKR
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIKPEAPGEDASPEELNRYYASLRHYLNLVTRQRYGKR

SEQ ID NO: 18 - protein, 214 aa, Homo sapiens
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 25 aa, Artificial Sequence
  G4Sx5
GGGGSGGGGSGGGGSGGGGSGGGGS

SEQ ID NO: 20 - protein, 273 aa, Artificial Sequence
  huIg-kappa-G4Sx5-PYY3-36
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSIKPEAPGEDASPEELNRYYASLRHYLNLVTRQRY

SEQ ID NO: 21 - protein, 274 aa, Artificial Sequence
  huIg-kappa-G4Sx5-PYY3-36-G
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSIKPEAPGEDASPEELNRYYASLRHYLNLVTRQRYG

SEQ ID NO: 22 - protein, 275 aa, Artificial Sequence
  huIg-kappa-G4Sx5-PYY3-36-GK
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSIKPEAPGEDASPEELNRYYASLRHYLNLVTRQRYGK

SEQ ID NO: 23 - protein, 276 aa, Artificial Sequence [sequence omitted]

(19) United States
(12) Patent Application Publication
(10) Pub. No.: US 2016/0333386 A1
(43) Pub. Date: Nov. 17, 2016

(54) USE OF PEPTIDYLGLYCINE ALPHA-AMIDATING MONOOXIGENASE (PAM) FOR C-TERMINAL AMIDATION

(71) Applicant: Hoffmann-La Roche Inc., Little Falls, NJ (US)

(72) Inventors: EIKE HOFFMANN, HERRSCHING a. AMMERSEE (DE); GEORG TIEFENTHALER, SINDELSDORF (DE)

(73) Assignee: Hoffmann-La Roche Inc., Little Falls, NJ (US)

(21) Appl. No.: 15/185,477

(22) Filed: Jun. 17, 2016

Related U.S. Application Data
(63) Continuation of application No. PCT/EP2014/077143, filed on Dec. 10, 2014.

(30) Foreign Application Priority Data
Dec. 20, 2013 (EP) 13199222.4

(51) Int. Cl. C12P 21/02

(52) U.S. Cl. CPC C12P 21/02; C12Y 114/17003; C12Y 403/02005

(57) ABSTRACT

One aspect as reported herein is a method for in vivo C-terminal amidation of a polypeptide characterized in that both the polypeptide (to be amidated) and human peptidylglycine alpha-amidating monooxigenase (PAM) are recombinantly co-expressed in a mammalian cell.

### CROSS REFERENCE TO RELATED APPLICATIONS

This application is a continuation of International Application No. PCT/EP2014/077143 having an international filing date of Dec. 10, 2014, the entire contents of which are incorporated herein by reference, and which claims benefit under 35 U.S.C. §119 to European Patent Application No. 13199222.4 filed Dec. 20, 2013.

### SEQUENCE LISTING

The instant application contains a Sequence Listing submitted via EFS-Web and hereby incorporated by reference in its entirety. Said ASCII copy, created on Jun. 15, 2016, is named P31912US1Seqlist.txt, and 61,892 bytes in size.

### FIELD OF INVENTION

The current invention is in the field of recombinant polypeptide production. Herein is reported a method for obtaining a C-terminally amidated polypeptide using human peptidylglycine alpha-amidating monooxigenase (PAM) in vivo.

### BACKGROUND OF THE INVENTION

In recent years the production of proteins has steadily increased and it is likely that proteins will become the biggest group of therapeutics available for the treatment of various diseases in the near future. The impact of proteins emerges from their specificity, such as the specific target recognition and binding function.

Cell cultures are used in fermentative processes to produce substances, in particular proteins. A distinction is made between processes in which the cell cultures are genetically unmodified and form their own metabolic products, and processes in which the organisms are genetically modified in such a manner that they either produce a larger amount of their own substances such as proteins, or produce substances which they do not produce without said modification, e.g. foreign (heterologous) substances.

More than half of the bioreactive neuropeptides and peptide hormones are amidated at their C-terminus. The synthesis normally occurs in endocrine, neuronal or other specifically differentiated secretory cells. The biosynthetic precursor for the amidated peptide is a C-terminally glycine-extended intermediate. The glycine-extended intermediate is usually generated from a larger precursor through an initial endoproteolytic cleavage at a processing site (generally composed of one or more basic amino acids). Thereafter the C-terminal basic residues are removed by a specific carboxypeptidase (for review see e.g. Bradbury, A. F. and Smyth, D. G., TIBS16 (1991) 112-115).

The (α-) amidating activity comprises two distinct enzymatic activities, a hydroxylase step and a lyase step mediated by a peptidyl-glycine α-amidating monooxygenase (PAM).

Wulf, B. S., et al. (Mol. Cell. Endocrin. 91 (1993) 135-141) report that efficient amidation of C-peptide deleted NPY precursors by non-endocrine cells is affected by the presence of Lys-Arg at the C-terminus. Tateishi, K. et al. (Biochem. Biophys. Res. Com. 205 (1994) 282-290) report the isolation and functional expression of human pancreatic peptidylglycine alpha-amidating monooxigenase. Takahashi, K.-Y. et al. (Peptides 18 (1996) 439-444) report the production of bioactive Salmon Calcitonin from COS-7 and CHO cells. Cloning, co-expression with an amidating enzyme, and activity of the scorpion toxin Bmk ITa1 cDNA in insect cells is reported by Liu, Z., et al. (Mol. Biotechnol. 24 (2003) 21-26). Manabu Satani et al. (Protein Express Purif. 28 (2003) 293-302) describe the expression and characterization of human bifunctional peptidylglycine alpha-amidating monooxigenase. C-terminal α-amidation is reported by Nozer M. Metha et al. (Post-translational modification of protein pharmaceuticals (2009) 253-276).

### SUMMARY OF THE INVENTION

It has been found that it is advantageous to use a certain ratio of (to be amidated) polypeptide encoding nucleic acid to PAM encoding nucleic acid to achieve a beneficial ratio of C-terminal amidation to yield of produced polypeptide.

Furthermore, is was found to not be a difference with respect to amidation and yield, if a membrane associated version of PAM was used (PAM2) or a transmembrane domain-depleted, soluble version of PAM (PAM 3).

One aspect as reported herein is a method for in vivo C-terminal amidation of a polypeptide characterized in that both the polypeptide (to be amidated) and human peptidylglycine alpha-amidating monooxigenase (PAM) are recombinantly co-expressed (co-expressed in a recombinant manner) in a mammalian cell.

One aspect as reported herein is a method for the recombinant production of a C-terminally amidated polypeptide characterized in that both the polypeptide and human peptidylglycine alpha-amidating monooxigenase (PAM) are recombinantly co-expressed (co-expressed in a recombinant manner) in a mammalian cell.

In one preferred embodiment of all aspects the human peptidylglycine alpha-amidating monooxigenase (PAM) is a PAM 3 (SEQ ID NO: 02).

In one embodiment of all aspects the mammalian cell is co-transfected with a first vector comprising an expression cassette comprising a nucleic acid encoding the polypeptide (to be amidated) and a second vector comprising an expression cassette comprising a nucleic acid encoding the PAM.

In one embodiment of all aspects the ratio of the first vector to the second vector is from about 90:10 to about 40:60. In one embodiment of all aspects the ratio of the first vector to the second vector is from about 70:30 to about 60:40. In one preferred embodiment of all aspects the ratio of the first vector to the second vector is from about 70:30 to about 60:40 and the human peptidylglycine alpha-amidating monooxigenase (PAM) is a PAM 3 (SEQ ID NO: 02).

In one embodiment of all aspects the mammalian cell comprises a first nucleic acid encoding the polypeptide and a second nucleic acid encoding the PAM.

In one embodiment of all aspects the ratio of the first nucleic acid to the second nucleic acid is from about 90:10 to about 40:60. In one embodiment of all aspects the ratio of the first nucleic acid to the second nucleic acid is from about 70:30 to about 60:40. In one preferred embodiment of all aspects the ratio of the first nucleic acid to the second nucleic acid is from about 70:30 to about 60:40. and the human peptidylglycine alpha-amidating monooxigenase (PAM) is a PAM 3 (SEQ ID NO: 02).

In one embodiment of all aspects a first mammalian cell comprising a nucleic acid encoding the polypeptide and a second mammalian cell comprising a nucleic acid encoding the PAM is used for co-expression.

In one embodiment of all aspects the ratio of the first mammalian cell to the second mammalian cell is from about 90:10 to about 40:60. In one embodiment of all aspects the ratio of the first mammalian cell to the second mammalian cell is from about 70:30 to about 60:40.

In one embodiment of all aspects the polypeptide is fused to the C-Terminus of an antibody heavy chain or the Fc region thereof.

In one embodiment of all aspects the polypeptide is Neurokinin, Allatostatin, Lem-KI, TRH, Red Pigment Concentrating Hormone, Calcitonin, CRF, LHRH, Leucopyrokinin, Gastrin I, Pigment Dispersing Hormone, Dermorphin, Oxytocin, Substance P, NPY, FMRFamide, Bombesin, Amylin, [Arg⁸]Vasopressin, BId-GrTH, Calcitonin, Cam-HrTH-II, Gastrin Releasing Peptide, Neuromedin B, Pancreastatin, Conotoxin M1, Secretin, GHRF, Melittin, Sarcotoxin 1A, VIP, α-MSH or MIF-1. In one embodiment of all aspects the polypeptide is peptide YY (PYY 3-36) of SEQ ID NO: 05.

One aspect as reported herein is a use of a human peptidylglycine alpha-amidating monooxigenase (PAM) for the recombinant production of a C-terminally amidated polypeptide, characterized in that both the polypeptide (to be amidated) and the human PAM are co-expressed in a recombinant manner in a mammalian cell.

### BRIEF DESCRIPTION OF THE SEQUENCES

SEQ ID NO: 01 Amino acid sequence of human PAM2
SEQ ID NO: 02 Amino acid sequence of human PAM3
SEQ ID NO: 03 Amino acid sequence of the human IgG1 Fc part
SEQ ID NO: 04 Amino acid sequence of G4Sx3 linker
SEQ ID NO: 05 Amino acid sequence of Peptide YY (PYY) 3-36
SEQ ID NO: 06 Amino acid sequence of Peptide YY (PYY) 3-36 plus glycine (G) at the C-terminus
SEQ ID NO: 07 Amino acid sequence of Peptide YY (PYY) 3-36 plus glycine (G) and lysine (K) at the C-terminus
SEQ ID NO: 08 Amino acid sequence of Peptide YY (PYY) 3-36 plus glycine (G) and lysine (K) and arginine (R) at the C-terminus
SEQ ID NO: 09 Amino acid sequence of fusion protein of a human IgG1 Fc part and a G4Sx3 linker and Peptide YY (PYY) 3-36
SEQ ID NO: 10 Amino acid sequence of fusion protein of a human IgG1 Fc part and a G4Sx3 linker and Peptide YY (PYY) 3-36 plus glycine (G) at the C-terminus
SEQ ID NO: 11 Amino acid sequence of fusion protein of a human IgG1 Fc part and a G4Sx3 linker and Peptide YY (PYY) 3-36 plus glycine (G) and lysine (K) at the C-terminus
SEQ ID NO: 12 Amino acid sequence of fusion protein of a human IgG1 Fc part and a G4Sx3 linker and Peptide YY (PYY) 3-36 plus glycine (G) and lysine (K) and arginine (R) at the C-terminus
SEQ ID NO: 13 Amino acid sequence of a human IgG1 heavy chain SEQ ID NO: 14 Amino acid sequence of fusion protein of a human IgG1 heavy chain and a G4Sx3 linker and Peptide YY (PYY) 3-36
SEQ ID NO: 15 Amino acid sequence of fusion protein of a human IgG1 heavy chain and a G4Sx3 linker and Peptide YY (PYY) 3-36 plus glycine (G) at the C-terminus
SEQ ID NO: 16 Amino acid sequence of fusion protein of a human IgG1 heavy chain and a G4Sx3 linker and Peptide YY (PYY) 3-36 plus glycine (G) and lysine (K) at the C-terminus
SEQ ID NO: 17 Amino acid sequence of fusion protein of a human IgG1 heavy chain and a G4Sx3 linker and Peptide YY (PYY) 3-36 plus glycine (G) and lysine (K) and arginine (R) at the C-terminus
SEQ ID NO: 18 Amino acid sequence of a human kappa light chain
SEQ ID NO: 19 Amino acid sequence of G4Sx5 linker
SEQ ID NO: 20 Amino acid sequence of fusion protein of a human kappa light chain and a G4Sx5 linker and Peptide YY (PYY) 3-36
SEQ ID NO: 21 Amino acid sequence of fusion protein of a human kappa light chain and a G4Sx5 linker and Peptide YY (PYY) 3-36 plus glycine (G) at the C-terminus
SEQ ID NO: 22 Amino acid sequence of fusion protein of a human kappa light chain and a G4Sx5 linker and Peptide YY (PYY) 3-36 plus glycine (G) and lysine (K) at the C-terminus
SEQ ID NO: 23 Amino acid sequence of fusion protein of a human kappa light chain and a G4Sx5 linker and Peptide YY (PYY) 3-36 plus glycine (G) and lysine (K) and arginine (R) at the C-terminus

### DESCRIPTION OF THE FIGURE

FIG. 1 Yield of C-terminally amidated product

An IgG-Fc molecule bearing a PYY+Gly peptide at its C-terminus was expressed recombinantly together with varying proportions of PAM3 expression plasmid (see table 3). Expression products were analysed for efficiency of C-terminal processing of the Gly residue by mass spectrometry, and expression yield was determined by protein A chromatography. From these two parameters, yield of C-terminally amidated product was calculated using the formula: yield of amidated product=total yield×percent C-terminal amidation/100. Results are from 2 independent experiments. *30% PAM3 value is the average of the 2 experiments.

### DETAILED DESCRIPTION OF THE INVENTION

Herein is reported a method for obtaining a recombinantly expressed C-terminally amidated polypeptide using human peptidylglycine alpha-amidating monooxigenase (PAM) in vivo.

It has been found that the use of a certain ratio of (to be amidated) polypeptide encoding nucleic acid to PAM encoding nucleic acid is beneficial to achieve an improved yield of C-terminal amidation of a recombinantly produced polypeptide in vivo compared to a process without recombinant human PAM.

Additionally it was found that instead of the membrane associated PAM (PAM2; main naturally occurring form) a soluble, i.e. transmembrane domain-depleted, PAM (PAM 3) can be used.

The term “about” denotes that the thereafter following value is no exact value but is the center point of a range that is +/−10% of the value, or +/−5% of the value, or +/−2% of the value, or +/−1% of the value. If the value is a relative value given in percentages the term “about” also denotes that the thereafter following value is no exact value but is the center point of a range that is +/−10% of the value, or +/−5% of the value, or +/−2% of the value, or +/−1% of the value, whereby the upper limit of the range cannot exceed a value of 100%.

The term “biologically active polypeptide” as used herein refers to an organic molecule, e.g. a biological macromolecule such as a peptide, protein, glycoprotein, nucleoprotein, mucoprotein, lipoprotein, synthetic polypeptide or protein, that causes a biological effect when administered in or to artificial biological systems, such as bioassays using cell lines and viruses, or in vivo to an animal, including but not limited to birds or mammals, including humans. This biological effect can be but is not limited to enzyme inhibition or activation, binding to a receptor or a ligand, either at the binding site or circumferential, signal triggering or signal modulation. Biologically active molecules are without limitation for example immunoglobulins, or hormones, or cytokines, or growth factors, or receptor ligands, or agonists or antagonists, or cytotoxic agents, or antiviral agents, or imaging agents, or enzyme inhibitors, enzyme activators or enzyme activity modulators such as allosteric substances.

One aspect as reported herein is a method for in vivo C-terminal amidation of a polypeptide characterized in that both the polypeptide to be amidated and human peptidylglycine alpha-amidating monooxigenase (PAM) are recombinantly co-expressed (co-expressed in a recombinant manner) in a mammalian cell.

Many polypeptides require a C-terminal amidation for biological activity. Some examples of such polypeptides are Neurokinin, Allatostatin, Lem-KI, TRH, Red Pigment Concentrating Hormone, Calcitonin, CRF, LHRH, Leucopyrokinin, Gastrin I, Pigment Dispersing Hormone, Dermorphin, Oxytocin, Substance P, NPY, FMRFamide, Bombesin, Amylin, [Arg⁸]Vasopressin, BId-GrTH, Calcitonin, Cam-HrTH-II, Gastrin Releasing Peptide, Neuromedin B, Pancreastatin, Conotoxin M1, Secretin, GHRF, Melittin, Sarcotoxin 1A, VIP, α-MSH or MIF-1 In an organism the C-terminal amidation is made by a specialized mechanism present in specialized cells, usually endocrine cells. This mechanism is not as efficient, or even not present, in mammalian cells normally used for the recombinant production of polypeptides.

Thus, a polypeptide that would be endogenously C-terminally amidated is not obtained at all, or not obtained in sufficient quantity, in C-terminally amidated form when produced recombinantly in mammalian cells.

To solve this problem, normally, polypeptides are C-terminally amidated “in vitro” after recombinant production and at least partial purification. In such an in vitro method the to-be-amidated-polypeptide is i) chemically or enzymatically modified at the C-terminus after the polypeptide itself had been produced in a different process and ii) exposed to non-natural (harsh) conditions.

In contrast thereto in the method as reported herein, the recombinantly produced polypeptides are amidated C-terminally already “in vivo”, i.e. during or shortly after their expression within the cell or the cultivation medium. In the context of this invention this means that the polypeptides are produced and C-terminally amidated in the same mammalian host cell or in the culture in which they have been produced without prior purification and without the addition of further enzymes. Thus, the production is performed in a continuous/constant process without intermediate isolation (or purification) of the to-be-amidated-polypeptide before the amidation takes place, i.e. the polypeptide is expressed and amidated in the same/a single step. This is achieved by the co-expression of the nucleic acid encoding the polypeptide of interest and a nucleic acid encoding an enzyme that is capable of introducing a C-terminal amidation in the polypeptide of interest, both in a recombinant manner.

One exemplary enzyme that introduces a C-terminal amidation in polypeptides is human peptidylglycine alpha-amidating monooxigenase (PAM).

The term “human peptidylglycine alpha-amidating monooxigenase” or “human PAM” denotes a polypeptide that has two enzymatically active domains with catalytic activities: peptidylglycine alpha-hydroxylating monooxygenase (PHM) and peptidyl-alpha-hydroxyglycine alpha-amidating lyase (PAL). The enzyme has two enzymatically active domains with catalytic activities. These catalytic domains work sequentially to transform neuroendocrine peptides to active alpha-amidated products.

Different splice variant (i.e. alternatively spliced transcripts) encoding different isoforms of PAM have been described. Two of these splice variants are the so-called PAM2 and PAM3 variants. The difference between the PAM2 and PAM3 transcripts is the presence, (PAM2) or absence, (PAM3) of the exons encompassing the transmembrane domain.

In one embodiment of all aspects the human peptidylglycine alpha-amidating monooxigenase (PAM) is a PAM 3 (SEQ ID NO: 02).

TABLE
Comparison between PAM2 and PAM3 splice
variant expression constructs

| | % PAM | % Gly |
|---|---|---|
| | co-transfected | cleaved |
| IgG1-Fc-PYY + Gly | 0% | 21% |
| IgG1-Fc-PYY + Gly | 1% PAM2 | 35% |
| IgG1-Fc-PYY + Gly | 3% PAM2 | 40% |
| IgG1-Fc-PYY + Gly | 10% PAM2 | 49% |
| IgG1-Fc-PYY + Gly | 30% PAM2 | 60% |
| IgG1-Fc-PYY + Gly | 1% PAM3 | 36% |
| IgG1-Fc-PYY + Gly | 3% PAM3 | 42% |
| IgG1-Fc-PYY + Gly | 10% PAM3 | 50% |
| IgG1-Fc-PYY + Gly | 30% PAM3 | 57% |

An IgG-Fc molecule bearing a PYY+Gly peptide at its C-terminus was expressed recombinantly. Together with the IgG-Fc expression plasmid, a varying proportion of either PAM2 or PAM3 expression plasmids was co-transfected. Expression products were analyzed for C-terminal processing of the Gly residue by mass spectrometry.

The term “expression” as used herein refers to transcription and/or translation processes occurring within a cell. The level of transcription of a nucleic acid sequence of interest in a cell can be determined on the basis of the amount of corresponding mRNA that is present in the cell. For example, mRNA transcribed from a sequence of interest can be quantitated by RT-PCR or by Northern hybridization (see Sambrook et al., 1989). Polypeptides encoded by a nucleic acid of interest can be quantitated by various methods, e.g. by ELISA, by assaying for the biological activity of the polypeptide, or by employing assays that are independent of such activity, such as Western blotting or radioimmunoassay, using immunoglobulins that recognize and bind to the polypeptide (see Sambrook et al., 1989, supra).

The term “co-expression” or “co-expressed” as used herein denotes that two or more nucleic acids encoding different recombinant polypeptides are expressed simultaneously in the same host cell or in two or more host cells cultivated together (in the same culture). In the first case a single host cell comprises all nucleic acids encoding the different polypeptides (the polypeptide-to-be-amidated and PAM). In the second case each of the host cell comprises at least one nucleic acid encoding a recombinant polypeptide (either the polypeptide-to-be-amidated or the PAM). For example, in case two different recombinant polypeptides are to be expressed simultaneously, either one, i.e. a single, cell comprising two recombinant polypeptide encoding nucleic acids is used or two cells each comprising (exactly) one recombinant polypeptide encoding nucleic acid are used. The different recombinant polypeptide encoding nucleic acids are comprised in mono- or multicistronic expression cassettes. These can either be on the same expression plasmid or on different expression plasmids.

The person skilled in the art understands that the term “recombinant” or “recombinantly” describes the situation where the nucleic acid encoding the polypeptide which is recombinant has been transfected into a mammalian cell. This might not be an (exclusively) endogenous polypeptide but at least in part artificially inserted into the cell.

An “expression plasmid” is a nucleic acid providing all required elements for the expression of the comprised structural gene(s) in a host cell. The term “vector” is used synonymously for “plasmid” within this application. Typically, an expression plasmid comprises a prokaryotic plasmid propagation unit, e.g. for E. coli, comprising an origin of replication, and a selectable marker, an eukaryotic selection marker, and one or more expression cassettes for the expression of the structural gene(s) of interest each comprising a promoter, a structural gene, and a transcription terminator including a polyadenylation signal. Gene expression is usually placed under the control of a promoter, and such a structural gene is said to be “operably linked to” the promoter. Similarly, a regulatory element and a core promoter are operably linked if the regulatory element modulates the activity of the core promoter.

An “expression cassette” refers to a construct that contains the necessary regulatory elements, such as promoter and polyadenylation site, for expression of at least the contained nucleic acid in a cell.

A “promoter” refers to a nucleic acid, i.e. polynucleotide sequence, which controls transcription of a nucleic acid to which it is operably linked. A promoter may include signals for RNA polymerase binding and transcription initiation. The promoter(s) used will be functionable in the cell type of the host cell in which expression of the operably linked nucleic acid is contemplated. A large number of promoters including constitutive, inducible, and repressible promoters from a variety of different sources are well known in the art (and identified in databases such as GenBank). They are available as or within cloned polynucleotides (from, e.g., depositories such as ATCC as well as other commercial or individual sources). A “promoter” comprises a nucleotide sequence that directs the transcription of e.g. an operably linked structural gene. Typically, a promoter is located in the 5′ non-coding or 5′-untranslated region (5′UTR) of a gene, proximal to the transcriptional start site of a structural gene. Sequence elements within promoters that function in the initiation of transcription are often characterized by consensus nucleotide sequences. These sequence elements include RNA polymerase binding sites, TATA sequences, CAAT sequences, differentiation-specific elements (DSEs; McGehee, R. E., et al., Mol. Endocrinol. 7 (1993) 551), cyclic AMP response elements (CREs), serum response elements (SREs; Treisman, R., Seminars in Cancer Biol. 1 (1990) 47), glucocorticoid response elements (GREs), and binding sites for other transcription factors, such as CRE/ATF (O'Reilly, M. A., et al., J. Biol. Chem. 267 (1992) 19938), AP2 (Ye, J., et al., J. Biol. Chem. 269 (1994) 25728), SP1, cAMP response element binding protein (CREB; Loeken, M. R., Gene Expr. 3 (1993) 253-264) and octamer factors (see, in general, Watson et al., eds., Molecular Biology of the Gene, 4th ed., The Benjamin/Cummings Publishing Company, Inc. 1987, and Lemaigre, F. P. and Rousseau, G. G., Biochem. J. 303 (1994) 1-14). If a promoter is an inducible promoter, then the rate of transcription increases in response to an inducing agent. In contrast, the rate of transcription is not regulated by an inducing agent if the promoter is a constitutive promoter. Repressible promoters are also known. For example, the c-fos promoter is specifically activated upon binding of growth hormone to its receptor on the cell surface. Tetracycline (tet) regulated expression can be achieved by artificial hybrid promoters that consist e.g. of a CMV promoter followed by two Tet-operator sites. The Tet-repressor binds to the two Tet-operator sites and blocks transcription. Upon addition of the inducer tetracycline, the Tet-repressor is released from the Tet-operator sites and transcription proceeds (Gossen, M. and Bujard, H., Proc. Natl. Acad. Sci. USA 89 (1992) 5547-5551). For other inducible promoters including metallothionein and heat shock promoters, see, e.g., Sambrook, et al. (supra), and Gossen, M., et al., Curr. Opin. Biotech. 5 (1994) 516-520. Among the eukaryotic promoters that have been identified as strong promoters for high-level expression are the SV40 early promoter, adenovirus major late promoter, mouse metallothionein-I promoter, Rous sarcoma virus long terminal repeat, Chinese hamster elongation factor 1 alpha (CHEF-1, see e.g. U.S. Pat. No. 5,888,809), human EF-1 alpha, ubiquitin, and human cytomegalovirus immediate early promoter (CMV IE). An enhancer (i.e., a cis-acting DNA element that acts on a promoter to increase transcription) may be necessary to function in conjunction with the promoter to increase the level of expression obtained with a promoter alone, and may be included as a transcriptional regulatory element. Often, the polynucleotide segment containing the promoter will include enhancer sequences as well (e.g., CMV or SV40).

The term “cell” or “host cell” refers to a cell into which a nucleic acid, e.g. encoding a heterologous polypeptide, can be or is introduced/transfected. If two or more vectors comprising nucleic acids are introduced in the same cell simultaneously, this process is called “co-transfection”. The term “cell” includes both prokaryotic cells, which are used for propagation of plasmids, and eukaryotic cells, which are used for the expression of a nucleic acid. Preferably, the eukaryotic cells are mammalian cells. Preferably the mammalian cell is selected from the group of mammalian cells comprising CHO cells (e.g. CHO K1, CHO DG44), BHK cells, NS0 cells, SP2/0 cells, HEK 293 cells, HEK 293 EBNA cells, PER.C6® cells, and COS cells. As used herein, the expression “cell” includes the subject cell and its progeny. Thus, the words “transformant” and “transformed cell” include the primary subject cell and cultures derived there from without regard for the number of transfers. It is also understood that all progeny may not be precisely identical in DNA content, due to deliberate or inadvertent mutations. Variant progeny that have the same function or biological activity as screened for in the originally transformed cell are included.

As the capacity of the translational machinery of a cell used for the recombinant production of a polypeptide is limited, balance between the expression of the polypeptide to be amidated and the PAM has to be achieved. Additionally, the balance between total yield and percentage of amidation of the polypeptide-to-be-amidated has to be considered.

In one embodiment of all aspects the mammalian cell is co-transfected with a first vector comprising an expression cassette comprising a nucleic acid encoding the polypeptide to be amidated and a second vector comprising an expression cassette comprising a nucleic acid encoding the PAM.

In one embodiment of all aspects the ratio of the first vector to the second vector is from about 90:10 to about 40:60. In one embodiment of all aspects the ratio of the first vector to the second vector is from about 70:30 to about 60:40. In one preferred embodiment of all aspects the ratio of the first vector to the second vector is from about 70:30 to about 60:40 and the human peptidylglycine alpha-amidating monooxigenase (PAM) is a PAM 3 (SEQ ID NO: 02).

In one embodiment of all aspects the mammalian cell comprises a first nucleic acid encoding the polypeptide and a second nucleic acid encoding the PAM.

In one embodiment of all aspects the ratio of the first nucleic acid to the second nucleic acid is from about 90:10 to about 40:60. In one embodiment of all aspects the ratio of the first nucleic acid to the second nucleic acid is from about 70:30 to about 60:40. In one preferred embodiment of all aspects the ratio of the first nucleic acid to the second nucleic acid is from about 70:30 to about 60:40. and the human peptidylglycine alpha-amidating monooxigenase (PAM) is a PAM 3 (SEQ ID NO: 02).

In one embodiment of all aspects a first mammalian cell comprising a nucleic acid encoding the polypeptide and a second mammalian cell comprising a nucleic acid encoding the PAM is used for co-expression.

In one embodiment of all aspects the ratio of the first mammalian cell to the second mammalian cell is from about 90:10 to about 40:60. In one embodiment of all aspects the ratio of the first mammalian cell to the second mammalian cell is from about 70:30 to about 60:40.

Where different mammalian cells are used for co-expression, the first mammalian cell does not comprise a nucleic acid encoding the PAM and the second mammalian cell does not comprise a nucleic acid encoding the polypeptide.

These ratios can be reflected (as in the current examples) by way of a percentage. For example a ratio of 40:60 (first vector/first nucleic acid; polypeptide to second vector/second nucleic acid; PAM) would be reflected as 60% PAM. Likewise, ratios of 70:30 or 60:40 would be reflected as 30% PAM or 40% PAM, respectively.

TABLE
C-terminal amidation versus yield

| | % PAM | Yield | % Gly |
|---|---|---|---|
| | co-transfected | [μg/mL] | cleaved |
| IgG1-Fc-PYY + Gly | 0% | 84 | 21% |
| IgG1-Fc-PYY + Gly | 1% PAM3 | 67 | 36% |
| IgG1-Fc-PYY + Gly | 3% PAM3 | 60 | 42% |
| IgG1-Fc-PYY + Gly | 10% PAM3 | 50 | 50% |
| IgG1-Fc-PYY + Gly | 30% PAM3 | 50-73 | 57%-67% |
| IgG1-Fc-PYY + Gly | 40% PAM3 | 41 | 76% |
| IgG1-Fc-PYY + Gly | 60% PAM3 | 15 | 80% |

An IgG-Fc molecule bearing a PYY+Gly peptide at its C-terminus was expressed recombinantly together with a varying proportion of PAM3 expression plasmids. Expression products were analyzed for C-terminal processing of the Gly residue by mass spectrometry, and yield was determined by protein A chromatography. Results are from 2 independent experiments.

One aspect as reported herein is a method for the recombinant production of a C-terminally amidated polypeptide characterized in that both the polypeptide and human peptidylglycine alpha-amidating monooxigenase (PAM) are recombinantly co-expressed (co-expressed in a recombinant manner) in a mammalian cell. In general, recombinant production of a polypeptide is performed by transfection of nucleic acids, cultivation of cells, harvesting of cells and purification of the polypeptide.

“Antibody heavy chain” refers to one part of a native antibody. A native antibody is a naturally occurring immunoglobulin molecule with varying structures. For example, native IgG antibodies are heterotetrameric glycoproteins of about 150,000 Daltons, composed of two identical light chains and two identical heavy chains that are disulfide-bonded. From N- to C-terminus, each antibody heavy chain has a variable region (VH), also called a variable heavy domain or a heavy chain variable domain, followed by three constant domains (CH1, CH2, and CH3).

The term “Fc region” or “Fc part” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

In one embodiment of all aspects the polypeptide is fused to the C-Terminus of an antibody heavy chain or the Fc region thereof.

Neuropeptide Y receptors (NYR) are a class of G-protein coupled receptors which are activated by the closely related peptide hormones neuropeptide Y, peptide YY and pancreatic polypeptide.

Peptide YY (PYY), also known as peptide tyrosine tyrosine or pancreatic peptide YY3-36, is a peptide that in humans is encoded by the PYY gene. Peptide YY is related to the pancreatic peptide family by having 18 of its 36 amino acids located in the same positions as pancreatic peptide. The two major forms of peptide YY are PYY1-36 and PYY3-36, which have PP fold structural motifs. However, the most common form of circulating PYY immunoreactivity is PYY3-36, which binds to the Y2 receptor (NPY2R, Y2R).

In one embodiment of all aspects the polypeptide is Neurokinin, Allatostatin, Lem-KI, TRH, Red Pigment Concentrating Hormone, Calcitonin, CRF, LHRH, Leucopyrokinin, Gastrin I, Pigment Dispersing Hormone, Dermorphin, Oxytocin, Substance P, NPY, FMRFamide, Bombesin, Amylin, [Arg⁸]Vasopressin, BId-GrTH, Calcitonin, Cam-HrTH-II, Gastrin Releasing Peptide, Neuromedin B,

Pancreastatin, Conotoxin M1, Secretin, GHRF, Melittin, Sarcotoxin 1A, VIP, α-MSH, MIF-1. In one embodiment of all aspects the polypeptide is peptide YY (PYY 3-36) of SEQ ID NO: 05.

One aspect as reported herein is a use of a human peptidylglycine alpha-amidating monooxigenase (PAM) for the recombinant production of a C-terminally amidated polypeptide, wherein both the polypeptide (to be amidated) and the human PAM are recombinantly co-expressed (co-expressed in a recombinant manner) in a mammalian cell.

The following examples are provided to aid the understanding of the present invention, the true scope of which is set forth in the appended claims. It is understood that modifications can be made in the procedures set forth without departing from the spirit of the invention.

### Materials and Methods

### Recombinant DNA Techniques

Standard methods were used to manipulate DNA as described in Sambrook, J. et al.,

Molecular cloning: A laboratory manual; Cold Spring Harbor Laboratory Press, Cold Spring Harbor, N.Y., 1989. The molecular biological reagents were used according to the manufacturer's instructions.

### Gene and Oligonucleotide Synthesis

Desired gene segments were prepared by chemical synthesis at Geneart GmbH (Regensburg, Germany). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany).

### Example 1

### Generation of Expression Plasmids for the Recombinant Expression/Co-Expression of Human PAM2 and PAM3

The human PAM2 and PAM3 encoding genes comprising the human PAM signal peptide, the propeptide sequence, and the sequences coding for mature human PAM2 or PAM3, respectively, were obtained by chemical synthesis and cloned into a cDNA expression vector as described above. The expression plasmid for the transient expression of human PAM2 or PAM3 in HEK293 cells comprised besides the PAM cDNA an origin of replication from the vector pUC18, which allows replication of this plasmid in E. coli, and a beta-lactamase gene which confers ampicillin resistance in E. coli. The transcription unit of the PAM2 or PAM3 molecules thus comprises the following functional elements:
the immediate early enhancer and promoter from the human cytomegalovirus (P-CMV) including intron A,
a human heavy chain immunoglobulin 5′-untranslated region (5′UTR),
the PAM2 or PAM3 cDNA including the PAM signal peptide and propeptide, and
the bovine growth hormone polyadenylation sequence (BGH pA).

### Example 2

### Generation of Expression Plasmids for the Recombinant Expression of Antibody-PYY Fusion Proteins and Antibody Fragment-Based PYY Fusion Proteins

a) Generation of Plasmids for the Expression of Human Immunoglobulin Heavy Chain-Derived Fragments Based on the Human IgG1 Constant Region (huIgG1-Fc) with C-Terminally Fused Peptide Sequences

The human IgG1-based antibody fragment-encoding fusion gene comprising the human IgG1 Fc fragment consisting of a partial hinge region and the IgG1 CH2 and CH3 domains and the respective peptide sequence was assembled by fusing a DNA fragment coding for the respective human IgG1 Fc fragment as detailed above to a sequence element coding for the respective peptide sequence separated by a glycine-serine linker (G4Sx3). In order to allow enzymatic C-terminal amidation, the sequence coding for a single glycine (-G) residue, or a glycine-lysine dipeptide (-GK), or a glycine-lysine-arginine tripeptide (-GKR) was added to the C-terminal amino acid of the respective IgG-Fc-peptide fusion molecule if not already present. The expression plasmid for the transient expression of a human IgG1-Fc-based antibody heavy chain fragment fusion molecule in HEK293 cells comprised besides the human IgG1-Fc fusion molecule an origin of replication from the vector pUC18, which allows replication of this plasmid in E. coli, and a beta-lactamase gene which confers ampicillin resistance in E. coli. The transcription unit of the IgG1-Fc-based antibody heavy chain fragment fusion molecule thus comprised the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a human IgG1 Fc encoding nucleic acid,
a glycine-serine linker (G4Sx3) encoding nucleic acid
a peptide with C-terminal G or GK or GKR encoding nucleic acid
the bovine growth hormone polyadenylation sequence (BGH pA).
   b) Generation of Plasmids for the Expression of Immunoglobulin Heavy Chains Using the Human IgG1 Constant Region with or without C-Terminally Fused Peptide Sequences

The human IgG1 heavy chain fusion gene comprising the human IgG1 constant region (CH1, hinge, CH2, CH3), a V-heavy variable domain, and, in case of a peptide fusion molecule the respective peptide sequence is assembled by fusing a DNA fragment coding for the human IgG1 constant region to a sequence element coding for a VH variable region and, in case of a peptide fusion molecule the sequence element coding for the respective peptide sequence separated by a glycine-serine linker (G4Sx3) to its C-terminus. In order to allow enzymatic C-terminal amidation, a single glycine (-G) residue, or a glycine-lysine dipeptide (-GK), or a glycine-lysine-arginine tripeptide (-GKR) is added to the C-terminal amino acid of the respective IgG-Fc-peptide fusion molecule if not already present. The expression plasmid for the transient expression of a human IgG1 heavy chain-based antibody fusion molecule in HEK293 cells comprises besides the human IgG1 heavy chain fusion molecule an origin of replication from the vector pUC18, which allows replication of this plasmid in E. coli, and a beta-lactamase gene which confers ampicillin resistance in E. coli. The transcription unit of the antibody heavy chain thus comprises the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a human IgG1 heavy chain encoding nucleic acid,
optionally a glycine-serine linker (G4Sx3) encoding nucleic acid and a peptide with C-terminal G or GK or GKR encoding nucleic acid
the bovine growth hormone polyadenylation sequence (BGH pA).
   c) Generation of Plasmids for the Expression of Immunoglobulin Light Chains Using the Human Ig-Kappa Constant Region with or without C-Terminally Fused Peptide Sequences

The human kappa light chain encoding fusion gene comprising the human Ig-kappa constant region (C-kappa), a V-kappa variable domain, and, if required a respective peptide sequence is assembled by fusing a DNA fragment coding for the human Ig-kappa constant region to a sequence element coding for a V-kappa variable region and, if required a sequence element encoding the respective peptide sequence separated by a glycine-serine linker (G4Sx5) to its C-terminus. In order to allow enzymatic C-terminal amidation, a single glycine (-G) residue, or a glycine-lysine dipeptide (-GK), or a glycine-lysine-arginine tripeptide (-GKR) is added to the C-terminal amino acid of the respective Ig-kappa-peptide fusion molecule. The expression plasmid for the transient expression of a human Ig-kappa-based antibody light chain fusion molecule in HEK293 cells comprises besides the human Ig-kappa fusion molecule an origin of replication from the vector pUC18, which allows replication of this plasmid in E. coli, and a beta-lactamase gene which confers ampicillin resistance in E. coli. The transcription unit of the antibody heavy chain thus comprises the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a human Ig kappa encoding nucleic acid,
optionally a glycine-serine linker (G4Sx5) encoding nucleic acid and a peptide with C-terminal G or GK or GKR encoding nucleic acid
the bovine growth hormone polyadenylation sequence (BGH pA).

### Example 3

### Transient Recombinant Expression of Antibody-PYY Fusion Proteins and Antibody Fragment-Based PYY Fusion Proteins

The recombinant fusion proteins were generated by transient transfection of HEK293 cells (human embryonic kidney cell line 293-derived) cultivated in F17 Medium (Invitrogen Corp.) with the respective expression plasmids. For transfection “293-Free” Transfection Reagent (Novagen) was used. The antibody- and antibody-based peptide-modified fusion molecules as described above were expressed from individual expression plasmids. For concomitant C-terminal amidation, PAM2- or PAM3-encoding expression plasmids were co-transfected together with the immunoglobulin expression plasmids. Transfections were performed as specified in the manufacturer's instructions. Recombinant protein-containing cell culture supernatants were harvested three to seven days after transfection. Supernatants were stored at reduced temperature (e.g. −80° C.) until purification. General information regarding the recombinant expression of human immunoglobulins in e.g. HEK293 cells is given in: Meissner, P. et al., Biotechnol. Bioeng. 75 (2001) 197-203.

### Example 4

### Purification of Recombinant Proteins

The Fc- or antibody fusion protein-containing culture supernatants were filtered and purified by two chromatographic steps. The fusion proteins were captured by affinity chromatography using MabSelectSuRe (GE Healthcare) equilibrated with PBS buffer, (10 mM Na₂HPO₄, 1 mM KH₂PO₄, 137 mM NaCl and 2.7 mM KCl, pH 7.4). Unbound proteins were washed out with equilibration buffer. The antibodies (or -derivatives) were eluted with 25-50 mM citrate buffer, pH 3.2. The protein containing fractions were neutralized with 0.1 ml 2 M Tris buffer, pH 9.0. Then, the eluted protein fractions were pooled, concentrated with an Amicon Ultra centrifugal filter device (MWCO: 10 K, Millipore) and loaded on a Superdex200 HiLoad 26/60 gel filtration column (GE Healthcare, Sweden) equilibrated with 20 mM histidine, 140 mM NaCl, at pH 6.0. The protein concentration of purified antibodies and derivatives was determined by determining the optical density (OD) at 280 nm with the OD at 320 nm as the background correction, using the molar extinction coefficient calculated on the basis of the amino acid sequence according to Pace et. al., Protein Science 4 (1995) 2411-2423. Monomeric Fc fractions were pooled, snap-frozen and stored at −80° C. Part of the samples was provided for subsequent protein analytics and characterization. Purity and proper formation of Fc- or antibody fusion proteins were analyzed by SDS-PAGE in the presence and absence of a reducing agent (5 mM 1,4-dithiotreitol) and staining with Coomassie brilliant blue. Aggregate content of the Fc-fusion protein preparations was determined by high-performance SEC using a GFC300 analytical size-exclusion column (Tosoh Bioscience, Stuttgart, Germany).

### Example 5

### FLIPR™ (Fluorescent Imaging Plate Reader) Assay

HEK-293 cells stably transfected with the G protein chimera Gαqi9 and the hygromycin-B resistance gene were further transfected with either Y2-receptor (Y2R) or the different human NPY receptors (NPY1-, NPY4- and NPY5-receptors) and G418 antibiotic selection. Following selection in both hygromycin-B and G418, individual clones were assayed for their response to PYY3-36. The transfected cells were cultured in DMEM medium supplemented with 10% fetal bovine serum, 50 μg/mL hygromycin-B, 2 mM glutamine, 100 U/mL penicillin, 100 μg/mL streptomycin and 250 μg/mL G418. Cells were harvested with trypsin-EDTA and counted using ViaCount reagent. The cell suspension volume was adjusted to 4.8×105 cells/mL with complete growth media. Aliquots of 25 μL were dispensed into 384-well Poly-D Lysine coated black/clear microplates (Falcon) and the microplates were placed in a CO₂ incubator overnight at 37° C. Loading buffer (Calcium-3 Assay Kit, Molecular Devices) was prepared by dissolving the contents of one vial (Express Kit) into 1000 mL Hank's Balanced Salt Solution containing 20 mM HEPES and 5 mM probenecid. Aliquots (25 μL) of diluted dye were dispensed into the cell plates and the plates are then incubated for 1 h at 37° C. During the incubation, test compounds were prepared at 3.5× the desired concentration in HBSS (20 mM HEPES)/0.05% BSA/1% DMSO and transferred to a 384-well plate for use on FLIPR™. After incubation, both the cell and compound plates were brought to the FLIPR™ and 20 μL of the diluted compounds were transferred to the cell plates by the FLIPR™. During the assay, fluorescence readings were taken simultaneously from all 384 wells of the cell plate every 1.5 seconds. Five readings were taken to establish a stable baseline, and then 20 μL of sample was rapidly (30 μL/sec) and simultaneously added to each well of the cell plate. The fluorescence was continuously monitored before, during and after sample addition for a total elapsed time of 100 seconds. Responses (increase in peak fluorescence) in each well following addition was determined. The initial fluorescence reading from each well, prior to ligand stimulation, was used as a zero baseline value for the data from that well. The responses are expressed as a percentage of maximal response of the positive control.

### Example 6

### PAM2 Versus PAM3

Several different splice variants of PAM are known to exist, two of these being the so-called PAM2 and PAM3 variants. The difference between the PAM2 and PAM3 transcripts is the presence (PAM2) or absence (PAM3) of the exons encompassing the transmembrane domain (Eipper et al., 1993). Thus, PAM is either inserted into the ER membrane (PAM2) or secreted into the ER lumen (PAM3). In order to assess the respective in vivo activity of co-transfected human PAM on C-terminal amidation, the human PAM sequences corresponding to the splice variants PAM2 and PAM3 were identified. cDNA segments encoding the respective human variants of PAM2 and PAM3 were prepared synthetically, and cloned into expression vectors as detailed above. Human IgG1-Fc-based molecules bearing a C-terminal peptide motif which was to be amidated (Fc-PYY+Gly) was expressed recombinantly. In addition to the Fc-PYY+Gly encoding plasmid, PAM2 or PAM3 encoding expression plasmids were co-transfected at different ratios to achieve amidation of the intermediate C-terminal Tyr residue in vivo in cell culture. The Fc fusion molecules were purified as described above and were subsequently analysed by mass spectrometry to assess the percentage of cleavage of the C-terminal Gly residue which was used as a measure for correct processing of the C-terminus by PAM and, consequently, the degree of amidation of the intermediate C-terminal Tyr residue, respectively, as detailed above. As shown in Table 1 up to about 60% (30% PAM co-transfected) of the C-terminal glycine residues were cleaved of the C-terminus posttranslationally in a dose dependent manner. No significant difference with regard to C-terminal Gly processing was seen between PAM2 and PAM3 constructs. All further experiments were conducted using PAM3 expression plasmids.

TABLE 1
Comparison between PAM2 and PAM3 splice
variant expression constructs

| | % PAM | % Gly |
|---|---|---|
| | co-transfected | cleaved |
| IgG1-Fc-PYY + Gly | 0% | 21% |
| IgG1-Fc-PYY + Gly | 1% PAM2 | 35% |
| IgG1-Fc-PYY + Gly | 3% PAM2 | 40% |
| IgG1-Fc-PYY + Gly | 10% PAM2 | 49% |
| IgG1-Fc-PYY + Gly | 30% PAM2 | 60% |
| IgG1-Fc-PYY + Gly | 1% PAM3 | 36% |
| IgG1-Fc-PYY + Gly | 3% PAM3 | 42% |
| IgG1-Fc-PYY + Gly | 10% PAM3 | 50% |
| IgG1-Fc-PYY + Gly | 30% PAM3 | 57% |

An IgG-Fc molecule bearing a PYY+Gly peptide at its C-terminus was expressed recombinantly. Together with the IgG-Fc expression plasmid, a varying proportion of either PAM2 or PAM3 expression plasmids was co-transfected. Expression products were analysed for C-terminal processing of the Gly residue by mass spectrometry.

### Example 7

### Analytical Characterisation of the C-Terminally Amidated Fusion Molecules

To determine the degree of C-terminal Tyr amidation, human IgG1-Fc-based molecules with different N-termini but identical C-termini (Fc-PYY+Gly) were expressed recombinantly. In addition to the Fc-PYY encoding plasmids, PAM3 expression plasmids (40% of total plasmid) were co-transfected to achieve improved amidation of the thus newly generated C-terminal Tyr residue in vivo in cell culture. The Fc fusion molecules were purified as described above and were subsequently analysed by mass spectrometry and peptide map analysis to assess the percentage of cleavage of the C-terminal Gly residue and the degree of amidation of the newly generated C-terminal Tyr residues, respectively. The integrity of the amino acid backbone of PYY fusion proteins was verified after removal of N-glycans by enzymatic treatment with peptide-N-glycosidase F (Roche Applied Science) by Electrospray ionization (ESI) mass spectrometry with and without prior reduction. Reduction was performed using TCEP. Desalting was performed on self-packed G25-Sephadex-Superfine columns using an isocratic formic acid gradient. ESI mass spectra (+ve) were recorded on a Q-TOF instrument (maXis, Bruker) equipped with a nano ESI source (TriVersa NanoMate, Advion). MS parameter settings were as follows: Transfer: Funnel RF, 400 Vpp; ISCID Energy, 0 eV; Multipole RF, 400 Vpp; Quadrupole: Ion Energy, 3.0 eV; Low Mass, 850 m/z; Source: Dry Gas, 8 L/min; Dry Gas Temperature, 160° C.; Collision Cell: Collision Energy, 8 eV; Collision RF: 3800 Vpp; Ion Cooler: Ion Cooler RF, 800 Vpp; Transfer Time: 140 μs; Pre Puls Storage, 20 μs; scan range m/z 600 to 2000. The MassAnalyzer software (developed in-house) was used for data evaluation. In addition, the degree of the cleavage of the C-terminal Gly residue relative to the full-length chain was deduced from the ESI mass spectra as this served as a first measure for the degree of C-terminal amidation since the Gly residue is removed during the enzymatic amidation process by PAM. In addition, the C-terminal amidation of the Tyr residue of those PYY fusion molecules with removed Gly was determined by peptide map analysis to prove also formally that the molecules lacking the C-terminal Gly residues possessed an amidated C-terminus. To this end, the PYY fusion proteins were reduced using DTT, alkylated using iodoacetic acid, and cleaved enzymatically using a combination of the proteases AspN and GluC (Roche Applied Science). Peptides were separated using reverse phase HPLC on a Polaris 3 C18 Ether column (Varian) and an acetonitrile/formic acid gradient. The effluent was split post column using a TriversaNanoMate, and a nanoliter flow portion was directed into the LC/MS interface and sprayed into an LTQ-FT mass spectrometer (Thermo) using electrospray ionization. The UV chromatograms at 220 nm and the ESI-MS and ESI-MS/MS were recorded. The C-terminal peptide lacking the C-terminal Gly residue was identified using the Mascot search algorithm (Matric science) and an in-house protein sequence database. The quantity of the amidated and the free acid form of the peptide were estimated relative to each other using the extracted ion chromatograms of the two species.

As shown in Table 2, between 63% and 86% of the C-terminal glycine residues were cleaved off the C-terminus posttranslationally; both mass spectrometry and peptide map analysis gave very similar results in this respect. In addition, peptide map analysis showed that over 98% of the molecules with a processed C-terminus (i.e. cleaved Gly residue) were modified by PAM and possessed an amidated C-terminus rather than an acidic C-terminus. Since, as detailed above, the C-termini of our recombinant molecules were found to be amidated almost quantitatively if the C-terminal Gly residue was cleaved, the degree of C-terminal Gly cleavage was used as a measure for correct processing (i.e. amidation) of the C-terminus in all future experiments.

TABLE 2
Percentage of different C-terminal modifications

| | | | Peptide map | | |
|---|---|---|---|---|---|
| | Mass spectrometry | | | % Gly | |
| | | % Gly | % Gly | cleaved | |
| | % Gly | not | cleaved | free | % Gly |
| | cleaved | cleaved | amidated C- | acid C- | not |
| | (total) | (total) | terminus | terminus | cleaved |
| IgG-Fc- | 76.6 | 23.4 | 78.5 | 0.4 | 21.1 |
| PYY + | | | | | |
| Gly | | | | | |
| GIP-G4S- | 63.0-86.3 | 13.7-37.0 | 64.5-84.6 | 0.8-1.2 | 14.2-34.7 |
| huIgG-Fc- | | | | | |
| PYY + | | | | | |
| Gly | | | | | |

Recombinant proteins produced using co-transfection of PAM3 expression plasmids were analysed for the presence or absence of a C-terminal Glycine residue using mass spectrometry (Gly cleaved or Gly not cleaved, columns 2 and 3), and the exact proportion of the different C-terminally modified species using peptide map analysis (Gly cleaved and amidated, Gly cleaved and not amidated, Gly not cleaved; columns 4-6).

### Example 8

### PAM Co-Expression Versus Yield

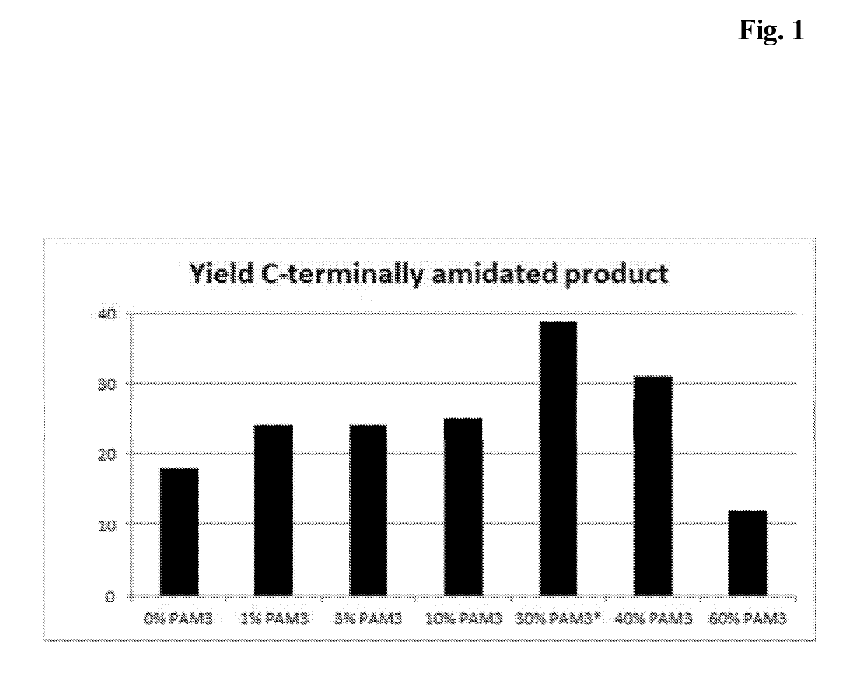

In addition to demonstrating equivalence between PAM2 and PAM3, the results shown in Table 1 also demonstrate C-terminal amidation of the PYY peptide moiety in a dose-dependent manner, i.e. the percentage of a processed C-terminus increases with an increased percentage of PAM expression plasmid in the transfection assay. In further experiments ratios between expression yield and C-terminal processing were determined. This was done using 1%, 3%, 10%, 30%, 40% or 60% PAM3 expression plasmid for co-transfection with the IgG1-Fc-PYY+Gly expression plasmid. C-terminal processing was higher when going up to 60% PAM3 expression plasmid as compared to 30%, (80% vs. 67% Gly processing). There was also a concomitant decrease in expression yield (15 μg/ml vs. 73 μg/ml). Both expression yield and C-terminal amidation have been assessed in combination (see FIG. 1). Based thereon, a percentage of 40% PAM expression plasmid was chosen for all future fermentations.

TABLE 3
C-terminal amidation versus yield

| | % PAM | Yield | % Gly |
|---|---|---|---|
| | co-transfected | [μg/mL] | cleaved |
| IgG1-Fc-PYY + Gly | 0% | 84 | 21% |
| IgG1-Fc-PYY + Gly | 1% PAM3 | 67 | 36% |
| IgG1-Fc-PYY + Gly | 3% PAM3 | 60 | 42% |
| IgG1-Fc-PYY + Gly | 10% PAM3 | 50 | 50% |
| IgG1-Fc-PYY + Gly | 30% PAM3 | 50-73 | 57%-67% |
| IgG1-Fc-PYY + Gly | 40% PAM3 | 41 | 76% |
| IgG1-Fc-PYY + Gly | 60% PAM3 | 15 | 80% |

An IgG-Fc molecule bearing a PYY+Gly peptide at its C-terminus was expressed recombinantly together with a varying proportion of PAM3 expression plasmids. Expression products were analysed for C-terminal processing of the Gly residue by mass spectrometry, and yield was determined by protein A chromatography. Results are from 2 independent experiments.

### Example 9

### Influence of C-Terminal Sequence on C-Terminal Processing

There had been a report in the literature that in addition to the Gly residue C-terminal of the amino acid to be amidated at its C-terminus which is required by the PAM enzyme for its activity, also the presence of the following two basic amino acids, namely Lys and Arg, affected the amidation of neuropeptide Y (NPY) by endogenous PAM of non-endocrine cells. Since PYY also possesses this sequence motif, -GlyLysArg, the influence of the presence or absence of this sequence motif was tested in combination with the co-expression of recombinant PAM3. As shown in Table 4, there was no significant difference with regard to C-terminal cleavage of the glycine residue. Consequently, peptide or protein sequences ending on -Gly, or GlyLys, or GlyLysArg can be viewed as equally effective with regard to C-terminal amidation by co-transfected recombinant PAM enzymes.

TABLE 4
Influence of C-terminal sequence
motif on processing of C-terminus

| | % PAM | % Gly |
|---|---|---|
| | co-transfected | cleaved |
| IgG1-Fc-PYY + G | no PAM3 | 17% |
| IgG1-Fc-PYY + G | 30% PAM3 | 68% |
| IgG1-Fc-PYY + G | 60% PAM3 | 79% |
| IgG1-Fc-PYY + GKR | no PAM3 | 24% |
| IgG1-Fc-PYY + GKR | 30% PAM3 | 76% |
| IgG1-Fc-PYY + GKR | 60% PAM3 | 79% |

IgG-Fc molecules bearing either a PYY+Gly peptide at its C-terminus or a PYY+GlyLysArg peptide at its C-terminus, were expressed recombinantly in combination with 30% or 60% PAM3 expression plasmids, or no PAM3 expression plasmids at all to establish endogenous baseline amidation. Expression products were analysed for C-terminal processing by mass spectrometry.

### Example 10

### Activity of In Vivo Amidated Recombinant Molecules on PYY Receptor

Recombinantly expressed human IgG1-Fc-based molecules bearing a C-terminal peptide motif which was to be amidated (Fc-PYY+Gly) was expressed, purified and analysed as detailed above, and tested in a cell culture assay using cells transfected with either the cognate receptor for PYY, Y2R, or the related receptors for NPY, namely NPY1R, NPY4R, or NPY5R as controls in a Ca-flux assay. Chemically synthesized PYY peptide bearing a tyrosine-amide residue at its C-terminus like the mature PYY molecule was used as a positive control, and similarly synthesized PYY peptide bearing a tyrosine residue with a carboxylic acid at its C-terminus was used as a negative control. As detailed in Table 5, the in vivo amidated Fc-PYY fusion molecules were clearly active with regard to stimulating the Y2R while simultaneously being inactive on the three different NPY-receptors (NPY1R, NPY4R, NPY5R). The molecules with a higher degree of a processed N-terminus tended to be more active than the molecules with a lower degree of N-terminal processing.

TABLE 5
Y2R activity of recombinant in vivo amidated Fc-PYY fusion molecules

| | | | Y2R | Y2R | | | |
|---|---|---|---|---|---|---|---|
| | % PAM | | EC50 | EC50 | NPY1R | NPY4R | NPY5R |
| | co- | % Gly | [nmol] | [nmol] | EC50 | EC50 | EC50 |
| | transfected | cleaved | Exp 1 | Exp 2 | [nmol] | [nmol] | [nmol] |
| PYY peptide- | — | — | 0.08 | — | 1862 | 42 | 214 |
| NH2 | | | | | | | |
| PYY peptide-OH | — | — | 57.1 | 21.3 | inactive | inactive | inactive |
| IgG1-Fc-PYY + G | no PAM3 | 21% | 0.64 | 3.3 | inactive | inactive | inactive |
| IgG1-Fc-PYY + G | 1% PAM3 | 36% | 0.71 | 0.73 | inactive | inactive | inactive |
| IgG1-Fc-PYY + G | 3% PAM3 | 42% | 0.64 | 0.47 | inactive | inactive | inactive |
| IgG1-Fc-PYY + G | 10% PAM3 | 50% | 0.44 | 0.51 | inactive | inactive | inactive |
| IgG1-Fc-PYY + G | 30% PAM3 | 57% | 0.29 | 0.37 | inactive | inactive | inactive |

1. A method for in vivo C-terminal amidation of a polypeptide characterized in that both the polypeptide and human peptidylglycine alpha-amidating monooxigenase (PAM) are recombinantly co-expressed in a mammalian cell.

2. A method for the recombinant production of a C-terminally amidated polypeptide characterized in that both the polypeptide and human peptidylglycine alpha-amidating monooxigenase (PAM) are recombinantly co-expressed in a mammalian cell.

3. The method according to any of the preceding claims, characterized in that the human peptidylglycine alpha-amidating monooxigenase (PAM) is a PAM 3 (SEQ ID NO: 02).

4. The method according to any of the preceding claims, characterized in that the mammalian cell comprises a first nucleic acid encoding the polypeptide and a second nucleic acid encoding the PAM.

5. The method according to claim 4, characterized in that the ratio of the first nucleic acid to the second nucleic acid is from about 90:10 to about 40:60.

6. The method according claim 4, characterized in that the ratio of the first nucleic acid to the second nucleic acid is from about 70:30 to about 60:40.

7. The method according to any of the preceding claims, characterized in that the polypeptide is fused to the C-Terminus of an antibody heavy chain or the Fc region thereof.

8. The method according any of the preceding claims, characterized in that the polypeptide is Neurokinin, Allatostatin, Lem-KI, TRH, Red Pigment Concentrating Hormone, Calcitonin, CRF, LHRH, Leucopyrokinin, Gastrin I, Pigment Dispersing Hormone, Dermorphin, Oxytocin, Substance P, NPY, FMRFamide, Bombesin, Amylin, [Arg⁸]Vasopressin, BId-GrTH, Calcitonin, Cam-HrTH-II, Gastrin Releasing Peptide, Neuromedin B, Pancreastatin, Conotoxin M1, Secretin, GHRF, Melittin, Sarcotoxin 1A, VIP, α-MSH or MIF-1.

9. The method according any of the preceding claims, characterized in that the polypeptide is peptide YY (PYY 3-36) of SEQ ID NO: 05.

10. Use of a human peptidylglycine alpha-amidating monooxigenase (PAM) for the recombinant production of a C-terminally amidated polypeptide, characterized in that both the polypeptide and the human PAM are recombinantly co-expressed in a mammalian cell.
